(19) United States
(12) Patent Application Publication
(10) Pub. No.: US 2015/0045782 A1
(43) Pub. Date: Feb. 12, 2015

(54) DISPENSING DEVICE FOR PRESSURIZED CONTAINERS FOR THE APPLICATION OF CRYOGENIC COOLANT

(71) Applicant: SIXTEM LIFE Srl, Sesto Fiorentino (IT)

(72) Inventor: Luciano Ottanelli, Sesto Fiorentino (IT)

(21) Appl. No.: 14/383,011

(22) PCT Filed: Jan. 11, 2013
(86) PCT No.: PCT/IB2013/050267

§ 371 Date: Sep. 4, 2014

(30) Foreign Application Priority Data
Mar. 9, 2012 (IT) PD2012A000071

(51) Int. Cl. A61J 1/14; B65D 83/28; A61B 18/02

(52) U.S. Cl. CPC A61J 1/1475; A61B 18/0218; A61J 1/1443; A61J 1/1412; B65D 83/285
USPC 606/25; 222/630

(57) ABSTRACT

The invention is a dispensing device for pressurized containers (C) of coolants for cryotherapy, comprising an opening/closing valve (C1), a dispenser body (A), a valve-opening element (B) positioned between said dispenser body (A) and said container (C) and resting on said valve (C1), a closing cap or plug (T) suited to be positioned on and removably constrained to said dispenser body (A) and comprising one or more teeth or projections (T21) resting on said valve-opening element (B) in order to open the valve (C1), a tube or duct (A1) included in and integral with said dispenser body (A), at least one applicator pad or filter (S) being partially inserted in a removable manner in one end of the tube or duct (A1).

This patent relates to medical devices for cryotherapy in the treatment of skin lesions and warts and in particular concerns a new dispensing device for pressurized containers for the application of cryogenic coolant.

Cryotherapy is a therapeutic procedure that uses fluids at extremely low temperatures for the localized treatment of skin lesions such as warts or other more or less serious skin diseases.

The controlled and precise application of the cooling fluid on the lesion leads to the freezing of intracellular water and the alteration of proteins and enzymes, causing, in the therapeutic application and subsequent defrosting time, dermo-epidermal detachment without damage to the dermis.

The advantages of cryotherapy are numerous. Firstly, anaesthesia of the area to be treated is not usually required and in addition, in most cases, scars do not appear and complex postoperative medications or treatments are not necessary. One of the fluids most used in cryotherapy is liquid nitrogen at a temperature of −196° C., while Freon, as a halogenated gas, has recently suffered restrictions as it is considered a contaminant.

The use of other fluids such as carbon dioxide or nitrous oxide is also known however they require more complex and expensive procedures and preparation and preservation precautions.

Currently, the cooling fluid to be used in cryotherapy is stored in pressurized containers with spray type dispensers with an opening/closing pressure valve, The known containers also comprise a removable tube or duct for application, at the end of which an applicator pad or swab suited to absorb and convey the coolant coming out of the container is placed.

The applicator pad or swab soaked in the cooling fluid is applied to the part to be treated, which is thus frozen, causing the rupture of the cell membranes and the subsequent dermo-epidermal detachment.

In the known containers, the tube or duct is applied to the container's dispenser in a removable manner at the time of use, that is, the user must first of all apply the tube, then place the applicator pad and finally dispense and apply the fluid. One of the drawbacks of the known devices is the fact that the uncontrolled release of the cooling fluid may occur, for example during the application or removal of the tube, resulting in a waste of material and, above all, the possibility of causing wounds and injuries to the user or the patient.

Furthermore, the operator must act quickly from the time the cooling fluid is dispensed to the time of application, to avoid an excessive change in the temperature of the fluid compared to the optimal values thereby reducing the effectiveness of the treatment.

Employing the known containers, comprising removable parts such as tubes and applicator pads, the operation often becomes laborious and therefore the operator acts with less promptness.

In addition, the operator must wait the appropriate technical times to make sure that the applicator pad is completely and sufficiently soaked in the cooling fluid before proceeding with the application.

To overcome the above drawbacks a new type of delivery device, for pressurized containers, has been designed and constructed for the application of cryogenic coolant gas in the therapeutic treatment of dermatological lesions. The main object of the present invention is to be able to be used with maximum safety for the operator and for the patient, because the uncontrolled release of the coolant fluid is hindered thanks to an activator cap or plug, which causes the fluid to be dispensed in an enclosed space to soak the applicator pad or filter.

Another object of the present invention is that it can be used in a simple, fast and immediate manner because the tube or duct to dispense the cooling fluid is integrated and integral with the dispenser body and therefore it is not necessary to assemble the tube ahead of time which inevitably results in a loss of time and the possible uncontrolled release of fluid.

The hygienic conditions are still guaranteed by the possibility of replacing the applicator pad or filter partially housed within the tube for each application.

Yet another object of the present invention is to speed up the application procedure, thereby also minimizing the variation of the fluid temperature before its application on the skin and thus increasing the efficiency of the cryotherapy, to the benefit of the patient,

One of the advantages of the present invention, resulting from the fact of using applicator pads or filters made of material with high cooling fluid absorption and conduction capacity, consists in the possibility of obtaining the immediate cooling of the application pad.

Another advantage of the present invention is that the operator is able to identify with certainty and timeliness the moment in which the applicator pad or filter is adequately soaked with coolant fluid and therefore ready for immediate application.

A further advantage is the fact that it is made with materials having suitable mechanical and chemical resistance to the substances used, as well as resistant to the low temperatures.

These and other aims, direct and complementary, are achieved by the new dispensing device for pressurized containers with a dispensing pressure valve for the application of cryogenic coolant gas in cryotherapy treatment, comprising a dispenser body with an integrated and integral tube, in which a cylindrical removable application pad or filter is partially inserted, suited to absorb and conduct the cooling fluid, and where a safety activator cap or plug is suited to close the dispenser body, preventing the accidental release of the fluid, and also to open the valve, leading to the controlled delivery of the fluid, which, conveyed through the tube, is immediately absorbed by the pad or filter. The activator cap comprises a casing suited to close the dispenser body, to which it is rigidly coupled in a removable manner, and an internal channel, integral with the upper internal part of the cap, suited to house the tube or duct of the dispenser body.

The cylindrical wall of the internal duct of the cap comprises, on the lower edge, one or more teeth or projections suitable to be inserted into corresponding slots or through holes on the dispenser body.

To dispense the fluid, the cap must be correctly positioned on the container and the container inverted, orienting it with the cap facing downwards.

Then acting on the cap with a force directed toward the dispenser body, the teeth or projections of the cap are totally or partially inserted in the slots and rest on an element, hereinafter called valve-opening element, positioned between the dispenser body and the upper edge of the container, connected to the container's valve.

The pressure applied on the valve-opening element, with the container inverted, causes the valve to open and, as a result of the force of gravity in addition to the pressure of the fluid in the pressurized container, results in the flow of fluid through the tube or duct integral with the dispenser body. The pad or filter partially inserted inside the tube absorbs the fluid and draws it up to the top, becoming completely soaked in the fluid.

Therefore, thanks to the combination of the forces of gravity and the pressure inside the container, the fluid flows out more rapidly, thereby considerably reducing the time needed for the complete soaking of the pad or filter.

As a result, the ideal application temperature can be reached in a shorter time. To maximize the absorption and conduction capacity of the cooling fluid, it is expected that the pad or filter is made of cellulose or open cell foam, preferably polyurethane S518.

The internal duct of the cap, which completely contains the tube, keeps the inserted pad or filter in the correct position, preventing it from being expelled due to the pressure of the gas.

Since the fluid is only dispensed with the container inverted and the cap facing downward, there is no risk of the uncontrolled release of any excess fluid not retained by the pad or filter,

Any such excess fluid, in fact, accumulates inside the upside down cap, where it evaporates gradually without being spilled outside.

This cap or plug is made of plastic material, having suitable mechanical, chemical and thermal resistance characteristics, and preferably transparent, so that the user, having activated the cap causing the fluid to be dispensed, can see from the outside whether the pad is soaked and if it is then ready to be used.

In fact, the flow of fluid and its absorption by the pad causes the deposition of condensate on the inner surface of the cap next to the pad itself, thus making it clear that the pad is soaked and ready for use.

On the cap and/or on the dispenser body and/or on the container there are ribs and/or grooves for the proper alignment and positioning of the cap.

In particular the cap can be aligned and positioned on the dispenser body in two different positions. In the closed “OFF” position the cap inserted on the dispenser body is rotated so that the teeth do not fit into the corresponding slots formed on the dispenser body and therefore it is not possible to trigger the dispensing of the fluid.

In the activated “ON” position, the cap is rotated for example by 180° relative to the closed “OFF” position, so that the teeth are aligned with the slots, therefore the user can, by pressing the cap towards the container, insert the teeth in the slots and act on the valve-opening element causing the fluid to be dispensed.

To guide the operator in the proper alignment of the cap in the closed or activated position, it is foreseen that the two correct positions are indicated on the cap itself, by means of cuts, symbols, or the like.

The characteristics of the new fluid dispensing device will be better clarified by the following description with reference to the drawings, attached by way of a non-limiting example.

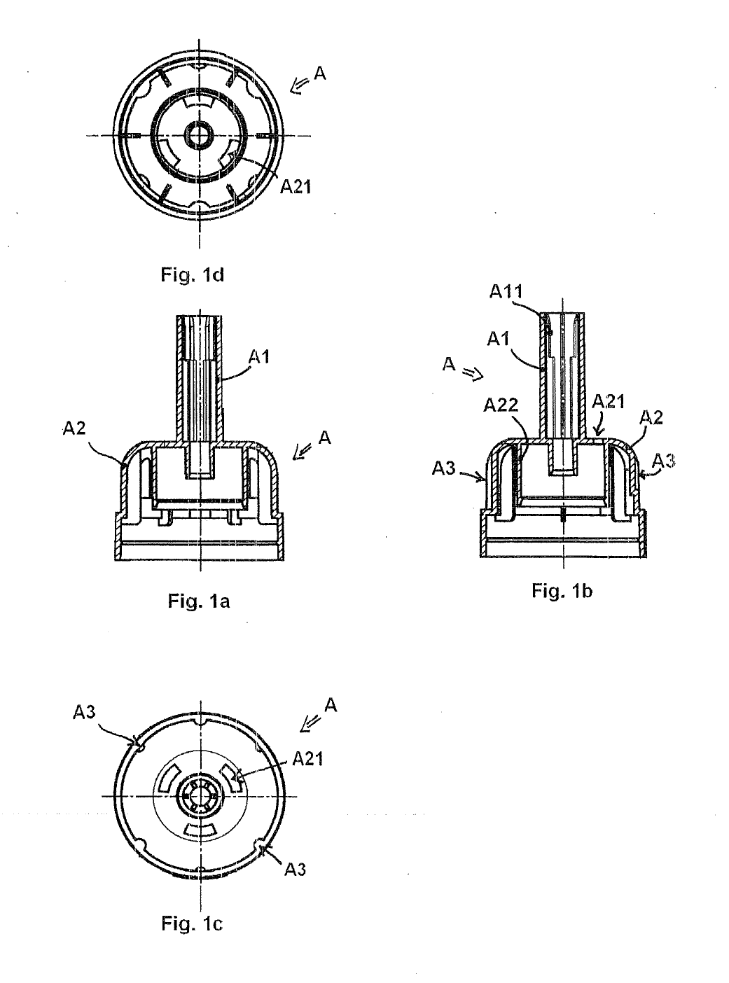

In FIGS. 1a-1d the dispenser body (A) is shown.

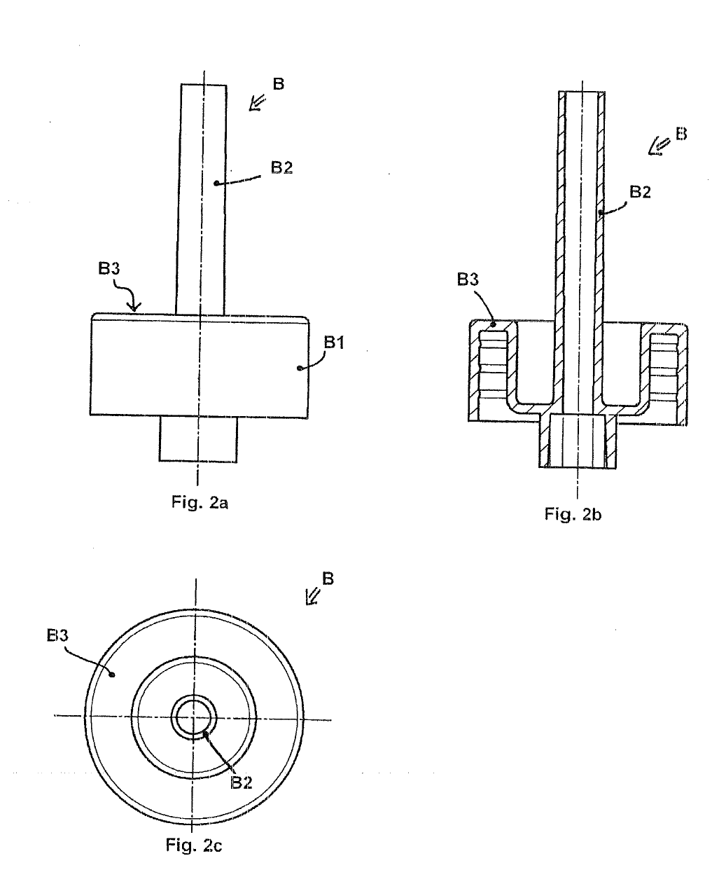

In FIGS. 2a-2c the valve-opening element (B) is shown.

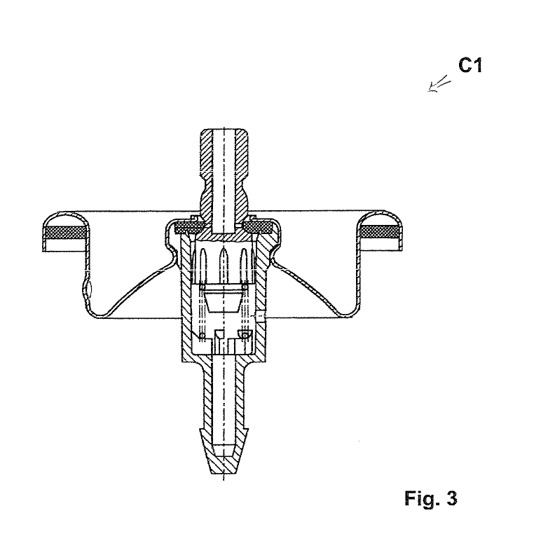

In FIG. 3 the valve (C1) of the pressurized container (C) is shown.

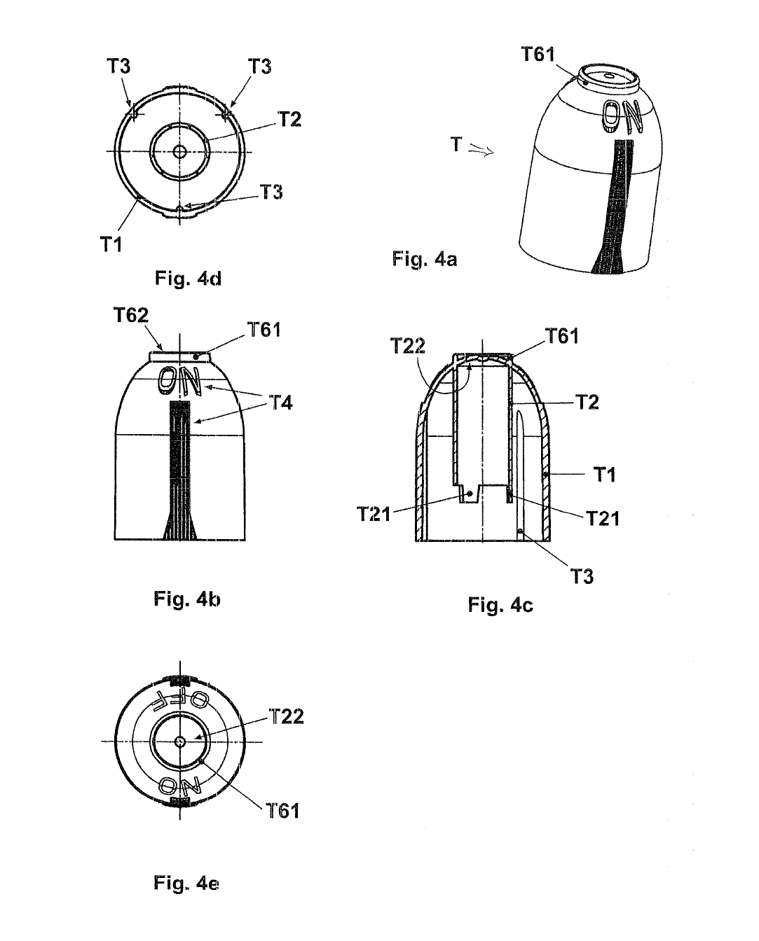

FIG. 4a shows a three-dimensional view of the activator cap (T), while

FIGS. 4b-4e show respectively a side view, a section, a bottom view and a top view.

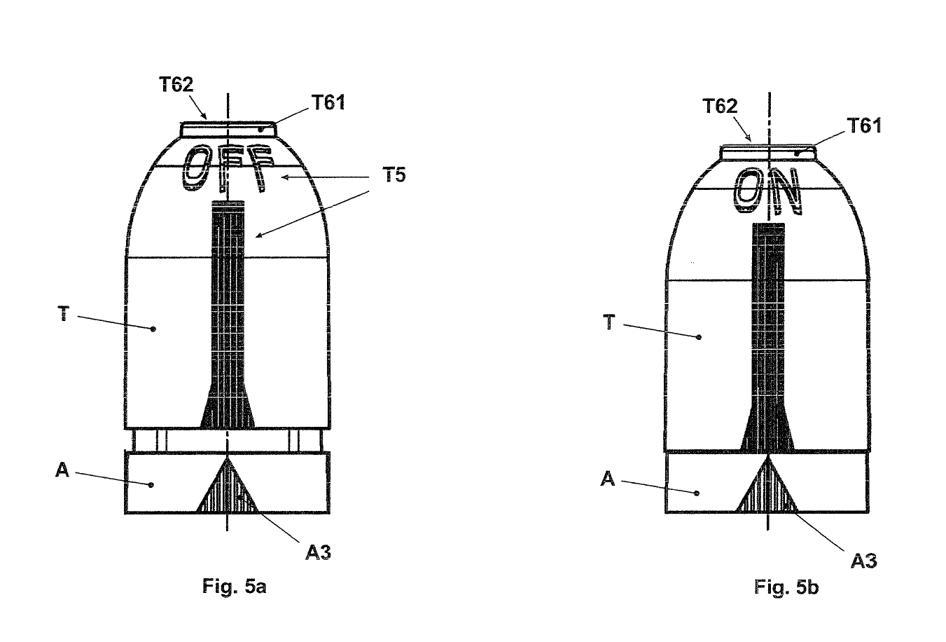

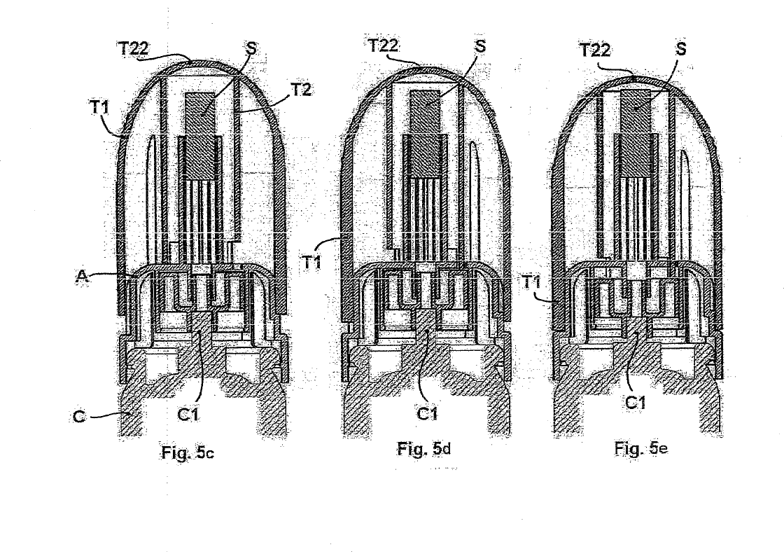

FIGS. 5a-5b schematically show the operation of the activator cap (T), which is shown in the closed “OFF” position in FIG. 5a and in the activated “ON” position in FIG. 5b.

FIGS. 5c-5e schematically show the cap (A) in section inserted on the dispenser body (A) in the different closed and activated positions.

The new dispensing device is applicable in an integral manner on pressurized containers (C) with a pressure valve (C1), of the spray type and comprising a dispenser body (A) suited to be fixed to the container (C).

The dispenser body (A) comprises a cup-shaped or convex part (A2) suited to contain the upper end of the container and at least one tube or duct (A1) integrated and integral with the cup-shaped part (A2), for the flow of cooling fluid stored in the container (C).

Said tube (A1) is internally shaped or comprises ribs (All) to hold at least one cylindrical pad or filter (S) partially inserted therein and shown in FIGS. 5c-5e.

In the preferred embodiment, to ensure the effective absorption and conduction of the cooling fluid, the cylindrical pad or filter (S) is made of polyurethane S518, with a diameter of about 7 mm and length of 18 mm.

Between the dispenser body (A) and the container (C) there is an interposed and fixed valve-opening element (B) shown in FIGS. 2a-2c.

The new dispensing device also comprises at least one activator cap or plug (T), shown schematically in FIGS. 4a-4e, comprising a casing (T1) suited to close the dispenser body (A), to which it is rigidly coupled in a removable manner, and an inner channel (T2), integral with the upper part of the cap (T), suited to house the tube (A1) of the dispenser body (A).

The cylindrical wall of said inner channel (T2) of the cap (T) comprises, on the lower edge, one or more teeth or projections (T21) suited to be inserted into corresponding slots or through holes (A21) formed on the cup-shaped part (A2) of the dispenser body (A), so as to rest on part (B3) of the valve-opening element (B), causing the opening of the valve (C1) of the container (C), shown in detail in FIG. 3, and thus the delivery of the fluid.

The fluid can only be dispensed with the container (C) inverted, that is with the cap (T) facing downward, and therefore the dispensing occurs, upon activation of the cap (T), as a result of the force of gravity and the pressure inside the container (C).

The fluid flow and its absorption by the pad (S) causes the deposition of condensate on the inner surface of the cap (T), near the upper part (T22) next to the pad (S) itself, thus making it clear to the user that the pad is soaked.

It is also foreseen that on the internal surface of the casing (T1) of the cap (T) there are one or more ribs (T3), suited to be inserted into corresponding grooves (A3) formed on the dispenser body (A), that guide the correct alignment and positioning of the cap (T).

In particular the cap can be aligned and positioned on the dispenser body in two different positions. In the closed “OFF” position (FIGS. 5a, 5c) the cap (T) inserted on the dispenser body is rotated so that the teeth (T21) are not aligned with the corresponding slots (A21) formed on the dispenser body (A) and therefore cannot be inserted to act on said valve-opening element (B).

In this case the fluid is prevented from being dispensed. When the cap (T) is in the closed “OFF” position, therefore, the device cannot be accidentally activated, thereby preventing the improper use by children or people in general. Furthermore, in the “OFF” position, the cap (T) is nonetheless bound to the dispenser body (A) and therefore also has a hygienic protection function pre- and post-use. That is, it protects the device from dust, germs and contaminants, ensuring adequate sanitary conditions until the next use.

In the activated “ON” position (FIGS. 5b, 5e), the cap (T) is rotated by 180° relative to the closed “OFF” position, so that the teeth (T21) are aligned correspondingly to the slots (A21).

When direct force is exerted on the cap (T) towards the container (C), the teeth (T21) slide into the slots (A21) and, resting on the valve-opening element (B), open the valve (C1) and dispense the fluid.

To guide the operator to the proper alignment of the cap in the closed/activated position, there is an indication on the cap (T) and on the dispenser body (A) regarding the proper alignment of the cap (T) to locate the closed “OFF” and activated “ON” positions.

In particular, it is foreseen that on the cap (T) there is an arrow and/or an indicator mark (T4) of the “ON” position and an arrow and/or an indicator mark (T5) of the “OFF” position, to align with the grooves (A3) indicated on the dispenser body (A).

To open the valve of the container and thus cause the fluid to flow, it is necessary to position the cap (T) in the activated “ON” position and orient the container with the cap facing downward, so that the fluid also flows out of the container due to the effect of the force of gravity.

To guide the user to the correct positioning of the container to dispense the fluid and to facilitate the activation of the cap (T), the cap (T) comprises on its upper part (T22) one or more ribs, protrusions or ridges in general (T61), for example arranged along a circumference with its centre coinciding with said upper part (T22) of the cap (T), suited to define a substantially plane surface (T62) for the stable support of the cap (T) on a plane surface.

Thus, after the successful activation of the cap (T) to dispense the fluid, the container may be left in the inverted position, with the cap (T) downwards to facilitate the complete soaking of the pad or filter (S),

Therefore, with reference to the preceding description and the attached drawings, the following claims are made.

1. Dispensing device for pressurized containers (C) of coolants for cryotherapy, comprising at least one manual opening/closing pressure valve (CI), characterized in that it comprises:
at least one dispenser body (A) suited to be permanently fixed to said container (C);
at least one valve-opening element (B) positioned between said dispenser body (A) and said container (C) and resting on said valve (CI);
at least one closing plug or cap (T) suited to be removably positioned on and fixed to said dispenser body (A) and comprising one or more teeth or projections (T21) resting on said valve-opening element (B) to cause the valve (CI) to open;
at least one tube or duct (A1) included in and integral with said dispenser body (A), at one end of which at least one applicator pad or filter (S) is removably inserted, characterized in that said cap (T), on its top (T5) opposite said container (C), comprises one or more projections, ribs or protrusions in general (T61) defining a substantially plane surface (T62) on which the cap (T) can firmly rest, and wherein, with the container overturned downwards, a force exerted on said cap (T) towards the dispenser body (A) makes said teeth or projections (T21) of the cap (T) press said valve-opening element (B), thus causing it to open and allowing the fluid to flow out of the container due to the force of gravity and/or to the pressure present in the pressurized container.

2. Dispensing device according to claim 1, characterized in that said cap (T) comprises an external casing (T1) that surrounds and encloses said dispenser body (A) and at least one internal channel (T2), integral with the upper part of the casing (T1), suitable for housing said tube (A1), and wherein the upper part (T22) of said cap (T) is in proximity to said pad or filter (S), partially inserted in said tube (A1), which is held in the correct position while the coolant is being dispensed.

3. Dispensing device according to claim 1, characterized in that said cap (T) is completely or partially made of a transparent plastic material that makes it possible to see from the outside the condensate that forms when the coolant is dispensed.

4. Dispensing device according to claim 1, characterized in that said valve-opening element (B) comprises at least one channel (B2) communicating with the opening of said valve (CI), at least partially inserted in said tube (A1) and suited to convey said dispensed coolant and make it reach said pad (S) partially inserted in said tube (A1).

5. Dispensing device according to claim 1, characterized in that said pad or filter (S) is made of cellulose or open-cell foam or another material with high capacity to absorb and convey the coolant.

6. Dispensing device according to claim 1, characterized in that said cap (T) and/or said dispenser body (A) and/or said container (C) are provided with one or more ribs (T3) and/or grooves (A3) for the guided application with correct alignment of said cap (T) to said dispenser body (A).

7. Dispensing device according to claim 1, characterized in that said dispenser body (A) comprises one or more through slots or holes (A21) for the insertion of one or more of said teeth or projections (T21) present on the lower edge of the internal channel (T2) of said cap (T), and wherein one or more of said teeth (T21), inserted through said slots (A21), rest on said valve-opening element (B) to cause the valve (CI) to be opened and the fluid to be dispensed.

8. Dispensing device according to claim 1, characterized in that said ribs (T3) and/or grooves (A3) allow said cap (T) to be applied to said dispenser body (A) and aligned in position “ON”, so that said teeth (T21) are aligned with said slots (A21) and can thus be inserted in them by pressing the cap (T) towards the container (C).

9. Dispensing device according to claim 1, characterized in that said ribs (T3) and/or grooves (A3) make it possible to guide said cap (T) into said dispenser body (A), aligning it in position “OFF”, rotated by a given angle with respect to said position “ON”, so that said teeth (T21) in position “OFF” are not aligned with said slots (A21) and thus cannot be inserted therein, and wherein in said closed position said cap (T) is constrained to said dispenser body (A) in order to protect it.

10. Dispensing device according to claim 1, characterized in that said cap (T) and/or said dispenser body (A) and/or said container (C) are provided with one or more cuts or symbols or reference marks (A3, T4, T5) for the correct alignment and arrangement of the cap (T) in position “OFF” or “ON”.
